(19) United States
(12) Patent Application Publication
(10) Pub. No.: US 2019/0162730 A1
(43) Pub. Date: May 30, 2019

(54) METHOD, COMPOSITION AND SENSOR FOR ANALYTE DETECTION

(71) Applicants: Cambridge Display Technology Limited, Godmanchester (GB); Sumitomo Chemical Company Limited, Tokyo (JP)

(72) Inventor: Jonathan Behrendt, Godmanchester (GB)

(21) Appl. No.: 16/309,880

(22) PCT Filed: Jun. 9, 2017
(86) PCT No.: PCT/GB2017/051689

§ 371 Date: Dec. 13, 2018

(30) Foreign Application Priority Data
Jun. 14, 2016 (GB) 1610347.5

(51) Int. Cl. G01N 33/66; G01N 33/52; G01N 31/22; C12Q 1/26

(52) U.S. Cl. CPC G01N 33/66; G01N 33/526; G01N 31/228; C12Q 2521/50; C12Y 101/03006; C12Y 101/03004; C12Q 1/26

(57) ABSTRACT

A method of testing a liquid sample for the presence of an analyte, the method comprising the steps of: forming a mixture by contacting the sample with a composition comprising an oxidase for formation of hydrogen peroxide from the analyte, a fluorescent indicator precursor capable of forming a fluorescent indicator in the presence of an oxygen radical and an iron compound wherein the iron compound is dissolved in the mixture; irradiating the mixture; and measuring fluorescence from the fluorescent indicator. The method may be carried out using a device in which the mixture in a channel or chamber (101) of a microfluidic device is irradiated by light from light source (103) and emission from the fluorescent indicator is detected by photodetector (105).

### FIELD OF THE INVENTION

The present invention relates to a method of detecting analytes by a fluorescent signal, compositions for producing said signal and sensors for carrying out said method.

### BACKGROUND

Fenton's reagent is a solution of hydrogen peroxide with an iron (II) compound that is used to form oxygen radicals by disproportionation of the iron (II) compound:

Fe²⁺+H₂O₂→Fe³⁺+HO.+OH⁻

Fe³⁺+H₂O₂→Fe²⁺+HOO.+H⁺

Woodward, J. et al. ‘Coupling of glucose oxidase and Fenton's reaction for a simple and inexpensive assay of beta-glucosidase’ Enzyme Microb. Technol. 1985, 7, 449-453 discloses an increase in absorption of ultraviolet light upon oxidation of ferrous sulfate to ferric sulfate. An assay of glucose oxidase and Fenton's reagent is proposed for measuring the activity of enzymes such as cellulose and beta-glucosidase.

Jiang, Y, et al, ‘Colorimetric detection of glucose in Rat Brain Using Gold Nanoparticles’ Angew. Chem. Int. Ed. 2010, 49, 4800-4804 discloses a gold nanoparticle-based assay for direct colorimetric visualisation of glucose in the rat brain based in a change in absorbance.

Hu, R. et al. ‘An efficient fluorescent sensing platform for biomolecules based on Fenton reaction triggered molecular beacon cleavage’ Biosens. Bioelectron. 2013, 41, 442-445 discloses a molecular beacon containing a fluorophore and a quencher. Hydroxyl radicals formed in-situ by action of glucose oxidase on glucose cleave the molecular beacon, causing separation of the fluorophore and the quencher.

Chih, T. et al. ‘Glucose sensing based on effective conversion of O₂ and H₂O₂ into superoxide anion radical with clay minerals’ J. Electroanal. Chem. 2005, 581, 159-166 discloses generation of superoxide anion radical from H₂O₂ and O₂ with montmorillonite K10 clay mineral, characterized by a fluorescence assay using amplex red and superoxide dismutase as probes.

It is an object of the invention to provide a method for detection of an analyte from which hydrogen peroxide may be formed that is capable of detection of the analyte at low concentrations.

It is a further object of the invention to provide a method for detection of an analyte from which hydrogen peroxide may be formed that is capable of detection of the analyte across a wide concentration range.

It is a yet further object of the invention to provide a low cost assay for detection of an analyte from which hydrogen peroxide may be formed.

### SUMMARY OF THE INVENTION

In a first aspect the invention provides a method of testing a liquid sample for the presence of an analyte, the method comprising the steps of:

forming a mixture by contacting the sample with a composition comprising an oxidase for formation of hydrogen peroxide from the analyte, a fluorescent indicator precursor capable of forming a fluorescent indicator in the presence of an oxygen radical and an iron compound wherein the iron compound is dissolved in the mixture;

irradiating the mixture; and

measuring fluorescence from the fluorescent indicator.

In a second aspect the invention provides a composition comprising an oxidase for formation of hydrogen peroxide from an analyte; an iron compound; and a fluorescent indicator precursor capable of forming a fluorescent indicator in the presence of an oxygen radical, wherein the fluorescent indicator precursor is selected from the group consisting of: fluoresceins, rhodamines, coumarins, boron-dipyrromethenes, naphthalimides, perylenes, benzanthrones, benzoxanthrones; and benzothiooxanthrones.

### DESCRIPTION OF THE DRAWINGS

The invention will now be described in more detail with reference to the figures in which:

FIG. 1A illustrates a sensor according to an embodiment of the invention comprising a light source and a photodetector on opposing sides of a microfluidic device;

FIG. 1B illustrates a sensor according to an embodiment of the invention comprising a light source and a photodetector on the same side of a microfluidic device;

FIG. 2 is a graph of sensor current vs. glucose concentration for mixtures formed according to an exemplary method of the invention having a relatively low iron concentration;

FIG. 3 is a graph of sensor current vs. glucose concentration for mixtures formed according to an exemplary method of the invention having a relatively high iron concentration; and

FIG. 4 is a graph of sensor current vs. time for mixtures formed according to an exemplary method of the invention having differing glucose oxidase concentrations.

### DETAILED DESCRIPTION OF THE INVENTION

The method described herein includes forming a mixture by bringing a liquid sample into contact with a composition comprising an iron compound, a fluorescent indicator precursor and an oxidase enzyme. The mixture may be formed by combining the liquid sample and the components of the composition in any order. Each component of the composition may be combined before being mixed with the liquid sample. The liquid sample may be mixed with one or more, but not all, components of the composition and then mixed with the remaining component or components of the composition.

The composition as described herein that is brought into contact with the liquid sample may be in solid form, optionally lyophilised form, or may be in a solution or suspension.

Upon contact of the composition with the sample, the following steps occur:

(i) oxidase-catalysed formation of hydrogen peroxide from the analyte compound;

(ii) formation of oxygen radicals by reaction of hydrogen peroxide with the iron compound; and

(iii) formation of a fluorescent indicator by reaction of the fluorescent indicator precursor with an oxygen radical.

“Oxygen radicals” as used herein means any species containing an oxygen radical atom, for example HO. or HOO.

Formation of Hydrogen Peroxide

The oxidase-catalysed formation of hydrogen peroxide may or may not require the presence of molecular oxygen (O₂). The reaction preferably occurs in an ambient air environment.

Hydrogen peroxide may be formed from the analyte by an oxidase-catalysed reaction of the analyte, or the analyte may undergo one or more preliminary reactions to form a compound capable of oxidase-catalysed production of hydrogen peroxide.

If the analyte undergoes one or more preliminary reactions then the or each reagent for the one or more preliminary reactions is preferably present in the composition. In this way, it will be appreciated that a cascade reaction consisting of one or more preliminary reactions and an oxidase-catalysed production of hydrogen peroxide may occur. Optionally, one or more reagents for the one or more preliminary reactions comprise at least one enzyme.

If hydrogen peroxide is formed by an oxidase-catalysed reaction of the analyte then the oxidase may be the only enzyme present in the composition.

Exemplary analytes and associated enzymes for production of hydrogen peroxide by an oxidase-catalysed reaction of the analyte include, without limitation: Glucose and glucose oxidase in the presence of molecular oxygen (O₂).

Cholesterol and cholesterol oxidase in the presence of molecular oxygen.

D-galactose and galactose oxidase in the presence of molecular oxygen.

D-amino acid and D-amino acid oxidase in the presence of molecular oxygen.

Hypoxanthine and xanthine oxidase in the presence of molecular oxygen.

L-gulono-1,4-lactone and L-gulonolactone oxidase in the presence of molecular oxygen.

An exemplary analyte that may undergo one or more preliminary reactions is a triglyceride, from which glycerol phosphate may be produced for oxidase-catalysed production of hydrogen peroxide by a glycerol phosphate oxidase-catalysed reaction in the presence of molecular oxygen. In this case, the assay optionally comprises a lipase for formation of glycerol from the triglyceride; and ATP and glycerol kinase for formation of glycerol phosphate by glycerol kinase-catalysed reaction of glycerol and ATP.

Another exemplary analyte is starch which may be hydrolysed to glucose via α-amylase and amyloglucosidase, from which H₂O₂ may be generated with glucose oxidase.

The concentration of the oxidase in the mixture of the composition and the liquid sample is optionally in the range of 0.5-200 μg/ml, optionally 1-100 μg/ml.

The oxidase enzyme, and any other reagents of the composition, are preferably dissolved in the mixture of the liquid sample and the composition.

Iron Compounds

An iron (II) or iron (III) compound, preferably an iron (II) compound, may be used in the mixture.

Hydrogen peroxide produced in situ by the oxidase catalysed reaction may react with iron (II) of an iron (II) compound present in the composition to form oxygen radicals.

The iron (II) compound may be any compound including, without limitation, an iron (II) salt, for example iron (II) sulfate or an iron (II) complex, for example iron (II) EDTA or iron (II) DTPA.

An iron (III) compound may be used in combination with catechol, for example as disclosed in “Degradation of recalcitrant compounds by catechol-driven Fenton reaction”, Water Science & Technology 49(4):81-4, February 2004.

The iron compound may be selected according to its desired solubility. The iron compound is preferably water soluble. Preferably, all iron ions of the composition are dissolved in the mixture formed from the composition and the liquid sample.

The iron ion concentration in the mixture is preferably at least 0.1 mM, more preferably at least 1 or at least 5 mM, and is optionally up to 50 mM.

Fluorescent Indicator Formation

The oxygen radicals formed by reaction of the hydrogen peroxide and iron compound may react with a fluorescent indicator precursor present in the assay to form the fluorescent indicator.

By “fluorescent indicator” as used herein is meant a material that fluoresces upon irradiation by light.

The presence of the fluorescent indicator may be measured by exciting the indicator with a light source and measuring fluorescence using a photodetector.

The presence of the analyte in the sample may be determined from the fluorescence measurement. If the analyte is present, its concentration in the sample may be determined.

The fluorescent indicator precursor emits little or no fluorescence upon irradiation with a light source, optionally a light source emitting light within the visible range (390-700 nm) or UV range (greater than 10 up to less than 390 nm, optionally 100-380 nm) as compared to the fluorescent indicator.

Preferably, the fluorescent indicator emits light upon irradiation with light in the visible range.

The fluorescent indicator precursor may be, without limitation, selected from the following compounds, each of which may be unsubstituted or substituted with one or more substituents: fluoresceins and salts thereof, rhodamines, coumarins, boron-dipyrromethenes (BODIPYs), naphthalimides, perylenes, benzanthrones, benzoxanthrones; and benzothiooxanthrones.

Exemplary substituents are chlorine, alkyl amino; phenylamino; and hydroxyphenyl. Exemplary fluoresceins include, without limitation, 2,7-dichlorofluorescein, 3′-(p-aminophenyl)fluorescein and 3′-(hydroyphenyl)fluorescein. A fluorescein indicator precursor may react with an oxygen radical to produce a fluorescent, oxidised fluorescein indicator.

The concentration of the fluorescent indicator precursor in the mixture of the composition and the liquid sample is optionally in the range of 0.1-10 mM, optionally 1-10 mM.

The fluorescein may have formula (Ia) or (Ib) or a salt thereof:

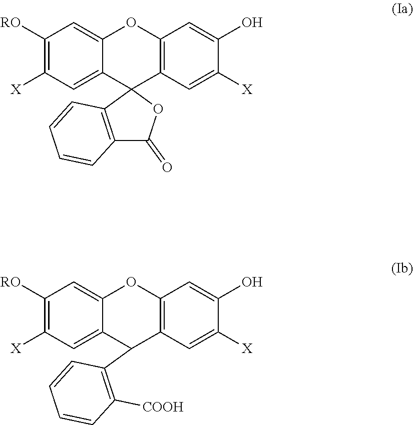

wherein X in each occurrence is independently H, F or Cl and R is H or a substituent, optionally phenyl which may be unsubstituted or substituted with one or more substituents. Substituents of phenyl may be hydroxyl or amino groups.

The fluorescent indicator precursor is preferably soluble in water. The fluorescent indicator precursor is preferably dissolved in the mixture.

Liquid Sample

The liquid sample as described herein is in the liquid state at ambient pressure (1 atmosphere) and ambient temperature (20° C.). It will be understood that the “liquid” sample may be, without limitation, a solution, a colloidal liquid or a suspension.

The liquid sample described herein may be a biological liquid, optionally blood, urine, saliva, tears, faeces, gastric fluid, bile, sweat, cerebrospinal fluid or amniotic fluid; cell culture media or other biological samples; or non-biological samples for example food, environmental water, e.g. river, sea or rain water, wine, or soil extracts.

Biological liquids may be analysed at physiological pH (ca. 7.4). Optionally, there is little or no effect on pH upon contact of the composition with a biological liquid. Optionally, any change in pH of the biological liquid upon contact with the composition is no more than 0.5, 0.2 or 0.1.

Analyte Detection

The method of detecting an analyte in a sample comprises the step of bringing a liquid sample into contact with a composition comprising or consisting of the iron compound, the fluorescent indicator precursor and the oxidase enzyme. Preferably, the composition does not comprise a quencher capable of quenching emission from the fluorescent indicator.

The liquid sample may be mixed with a solution or suspension of the composition or may be contacted with the composition in solid form, optionally lyophilised form.

The iron compound and the fluorescent indicator precursor are preferably in a dissolved form during analyte detection. If the liquid sample is mixed with a solution or suspension of the composition then the iron compound and the fluorescent indicator precursor are preferably dissolved in the solvent of the solution or suspension. If the liquid sample is contacted with the composition in solid form then the iron compound and the fluorescent indicator preferably dissolve in the liquid sample.

The oxidase may be dissolved in the solution or suspension.

The oxidase may be immobilised on a solid surface, optionally a polymer surface, in the solution or suspension or in the solid composition.

If the analyte is converted by one or more preliminary reactions to a compound capable of oxidase-catalysed production of hydrogen peroxide then the or each reagent for the one or more preliminary reactions may each independently be immobilised on a solid surface, dissolved in a solvent or provided in the composition in solid form.

The liquid sample may be brought into contact with the composition disposed in or on a device for mixing the liquid sample and the composition. The composition may be provided in a channel or chamber of a microfluidic device or immobilised on a surface of a lateral flow device.

The mixture is irradiated with a light source. Any light source may be used including, without limitation, an inorganic LED or LED array; one or more organic light-emitting devices (OLEDs); a laser; or an arc lamp. The light source is preferably an OLED.

OLEDs comprise an anode, a cathode and a light-emitting layer comprising an organic light-emitting material between the anode and the cathode. One or more further layers may be provided between the anode and the cathode, optionally one or more charge-transporting, charge injecting or charge-blocking layers. Upon application of a bias between the anode and cathode, light is emitted from the organic light-emitting material. OLEDs may be as described in Organic Light-Emitting Materials and Devices, Editors Zhigang Li and Hong Meng, CRC Press, 2007, the contents of which are incorporated herein by reference.

The fluorescent indicator preferably emits light upon irradiation of light in the visible range of 390-700 nm and the wavelength range of light emitted from the light source may be selected accordingly.

Light emitted from the fluorescent indicator is preferably in the visible range or in the infra-red range (greater than 700 nm, optionally at least 750 nm, up to about 1000 nm) preferably in the visible range.

Light emitted from the fluorescent indicator may be detected by a photodetector, optionally an organic photodetector (OPD), a charge-coupled device (CCD) or a photomultiplier, preferably an OPD or CCD.

An OPD comprises an anode, a cathode and an organic semiconducting region between the anode and cathode. The organic semiconducting region may comprise adjacent electron-donating and electron-accepting layers or may comprise a single layer comprising a mixture of an electron-accepting material and an electron-donating material. One or more further layers may be provided between the anode and the cathode. Conversion of light incident into electrical current may be detected in zero bias (photovoltaic) mode or reverse bias mode. OPDs may be as described in Ruth Shinar & Joseph Shinar “Organic Electronics in Sensors and Biotechnology” McGraw-Hill 2009, the contents of which are incorporated herein by reference.

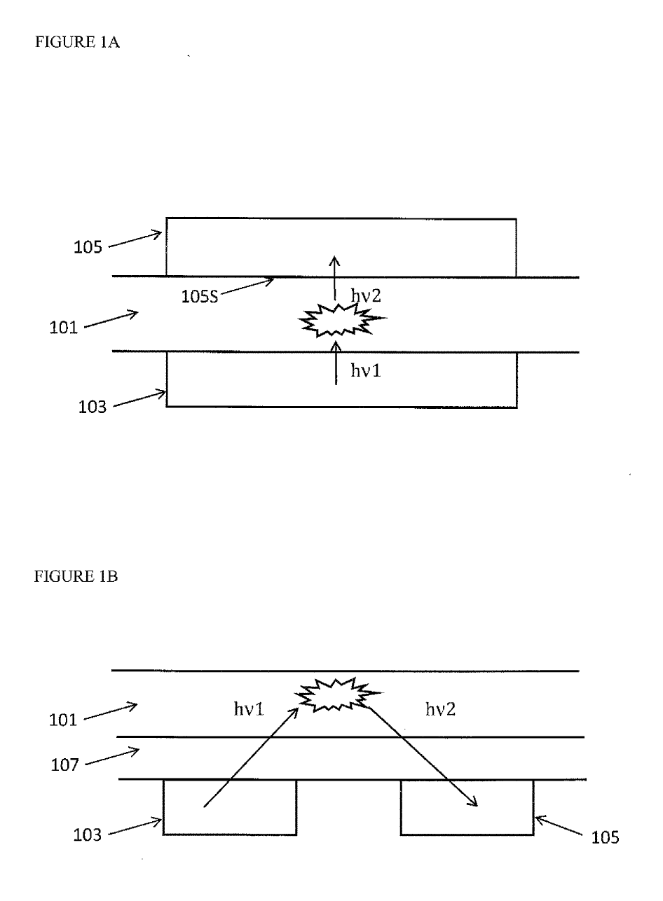

FIG. 1A, which is not drawn to any scale, illustrates a sensor suitable for use in a method as described herein comprising a light source, a photodetector and a microfluidic device.

In use, a liquid sample is contacted with the composition described herein in channel or chamber 101 of a microfluidic device and is illuminated with light from light source 103 of wavelength hv1. If the fluorescent indicator has been formed then the light from the light source is absorbed and re-emitted by the fluorescent indicator as light of longer wavelength hv2 which may be detected by photodetector 105 having a surface 105S on which light is incident.

In the embodiment of FIG. 1A, the light source 103 is provided on a first surface of the microfluidic device and the photodetector 105 is provided on an opposing, second surface.

A filter (not shown) may be provided between the light source and the photodetector to eliminate some or all wavelengths of light other than a wavelength range emitted by the fluorescent indicator.

A filter (not shown) may be provided between the light source and the mixture to eliminate some or all wavelengths of light other than a wavelength range absorbed by the fluorescent indicator.

FIG. 1B, which is not drawn to any scale, illustrates another sensor other arrangement in which the light source 103 and photodetector 105 are provided on a first surface of the microfluidic device. In this embodiment, light emitted from the light source may be prevented from reaching the photodetector 105 by use of a highly absorbing (black) layer on or over a second surface of the microfluidic device opposing the first surface and/or by use of a filter on or over the surface of the photodetector on which light is incident.

The light source 103 and photodetector 105 are provided on a common substrate 107, such as a glass or plastic substrate, provided adjacent to the first surface of the microfluidic device. In another embodiment, the first surface of a microfluidic device may form a common substrate on which the light source and photodetector are formed. In a yet further embodiment, light source 103 and photodetector 105 may be provided on separate substrates on the first surface.

In the case where the light source is an OLED and the photodetector is an OPD, the OLED and photodetector may be formed on a common substrate which is then brought adjacent to the first surface of the microfluidic device to form the sensor. The OPD and OLED of this embodiment may be formed using a common transparent anode layer on the substrate, optionally a common indium tin oxide layer.

It will be appreciated that the light source and photodetector may be provided in a wide range of arrangements to sense emission of fluorescent light from the fluorescent indicator and may be used with, without limitation, filters, light-absorbing layers, light-reflecting layers, lenses, optical fibres and combinations thereof.

The sensor may have a modular structure in which the microfluidic device is separable from the light source and/or photodetector. Optionally, the microfluidic device of the sensor comprises a single use glass or transparent plastic microfluidic chip which may be removed and replaced with another chip.

Optionally, the microfluidic device is not modular, the entire sensor being a single-use sensor.

The or each component of the composition may be introduced into a microfluidic device from a solution or suspension comprising one or more, optionally all, components of the composition dissolved or suspended therein and then lyophilising the solution or suspension.

The solid composition may be absorbed onto or into a lateral flow device by applying the components of the composition from one or more solutions or suspensions onto a surface of the device followed by evaporation of the solvent or solvents of the solution or suspension.

The sensor may be a portable device. The sensor may be a handheld device.

FIGS. 1A and 1B illustrate a sensor comprising a microfluidic device in which the sample is brought into contact with the composition, however it will be appreciated that other apparatus may be used for mixing the liquid sample with the composition, for example a lateral flow device having a surface on which the composition is immobilised in solid form.

FIGS. 1A and 1B illustrate a sensor having only one light source and only one photodetector. There may be more than one light source for each detector.

The sensor may be a multi-channel microfluidic device wherein at least one channel is configured to detect an analyte as described herein, the one or more further channels each being configured to detect a different analyte by a method as described herein or by another method known to the skilled person.

The sensors described herein may enable detection of analytes at low concentration and/or across a wide analyte concentration range. The analyte concentration in the sample for analysis may be in the range of about 1 pM-300 mM, optionally 0.1-100 mM, optionally 0.2-10 mM.

Applications

The compositions described herein may be used in an assay for detection of analytes including, without limitation, glucose, cholesterol, triglycerides and sensors as described herein may be used as point-of-care sensors for quantitative measurement of said analytes.

### EXAMPLES

All reagents were purchased from Sigma Aldrich.

### Example 1: Formation of 2,7-Dichlorofluorescin Detection Reagent

2,7-Dichlorofluorescin diacetate was dissolved in DMSO at a concentration of 1 mg/mL (2 mM). To 50 μL of this solution was added methanol (50 μL) and 2M aqueous potassium hydroxide (50 μL) and the mixture was left to stand at room temperature for 1 hour (final concentration of detection reagent is 0.67 mM).

### Example 2: Glucose Assay—Lower Iron (II) Concentration

Solutions were prepared containing the following: 15 μL of detection reagent solution (as prepared in Example 1), 100 μL aqueous solution of EDTA (2.5 mM), 100 μL aqueous solution of iron (II) sulfate (2.5 mM) and 685 μL solution of D-(+)-glucose (0.1, 0.3, 1, 3, or 10 mM) in sodium phosphate buffer (0.1 M, pH 7.4). To each of these solutions was added 100 μL solution of glucose oxidase (20 mg/mL) in water and the sample tube was rapidly inverted to mix. After 1 h, ˜130 μL of the solution was used to entirely fill a microfluidic flow cell (20×9 mm area with an optical pathlength of 0.5 mm).

This flow cell was placed in an OLED/OPD detector as illustrated in FIG. 1A having a short pass filter between the OLED and the microfluidic flow cell and a long pass filter between the microfluidic flow cell and the OPD.

The OLED was supported on a glass substrate and comprised a transparent anode, a hole injection layer, a polymeric hole-transporting layer, a light-emitting layer comprising a fluorescent blue light-emitting polymer and a cathode. The peak emission wavelength of the OLED was 480 nm.

The OPD was supported on a glass substrate and comprised a transparent anode, a hole transporting layer, a layer of a mixture of a donor polymer illustrated below and a C70 fullerene acceptor material and a cathode.

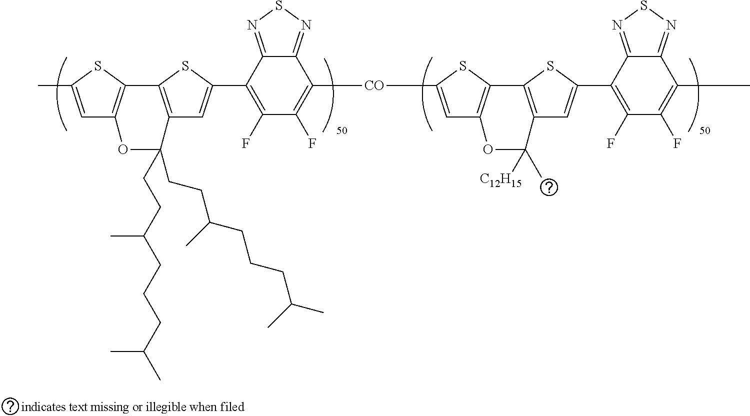

Fluorescence from the fluorescent indicator was measured used a drive current of 20 mA, an OPD bias of 0 V and a pulse time of 100 ms. The printed short pass and long pass filters were used to sharpen the OLED spectrum and prevent excitation light from reaching the OPD.

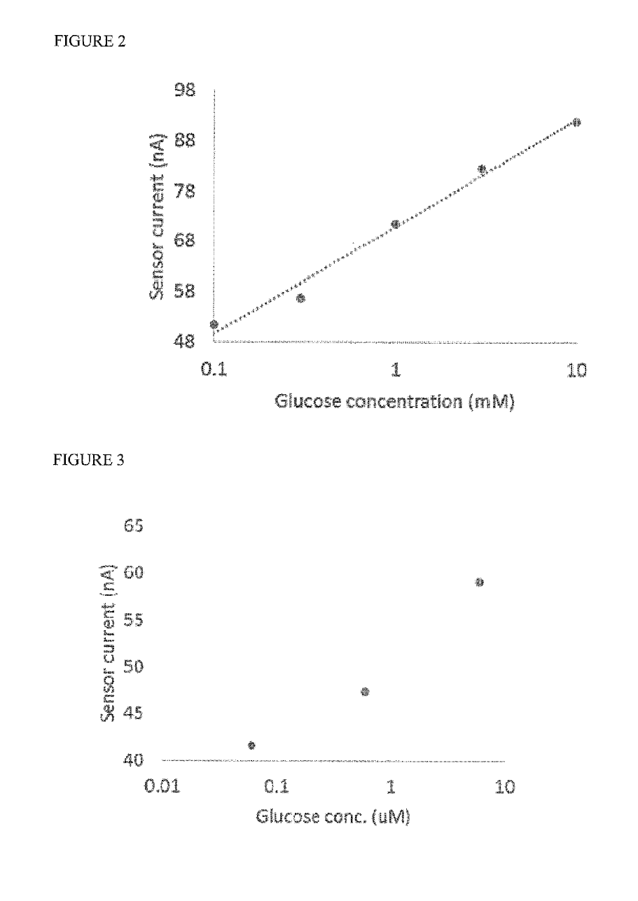

With reference to FIG. 2, there is a linear relationship between sensor current (corresponding to intensity of fluorescence from the fluorescent indicator) and concentration of glucose.

### Example 3: Glucose Assay—Higher Iron (II) Concentration

Solutions were prepared containing the following: 15 μL of detection reagent solution (as prepared in example 1), 50 μL aqueous solution of iron (II) sulfate (100 mM), 50 μL aqueous solution of EDTA, 785 μL of D-(+)-glucose (0, 0.06, 0.6 or 6 μM) in phosphate buffered saline (pH 7.4). To each of these solutions was added 100 μL solution of glucose oxidase (20 mg/mL) in water and the sample tube was rapidly inverted to mix. After 5 minutes at room temperature, ˜130 μL of the solution was used to entirely fill a microfluidic flow cell (20×9 mm area with an optical pathlength of 0.5 mm) and the fluorescence intensity was measured as described in Example 2.

With reference to FIG. 3, a substantially linear relationship was observed between glucose concentration and sensor current.

### Example 4

Three solutions were prepared as in Example 3. To each of these solutions was added a solution of glucose oxidase in water to give a final enzyme concentrations of 0.02, 0.2 or 2 mg/mL and a final volume of 1 mL. After mixing, ˜130 uL of solution transferred immediately to a microfluidic flow cell (20×9 mm area with an optical pathlength of 0.5 mm) and the fluorescence intensity was measured every 15 seconds over a 20 minute time course using the OLED/OPD platform and measurement parameters described in Example 2.

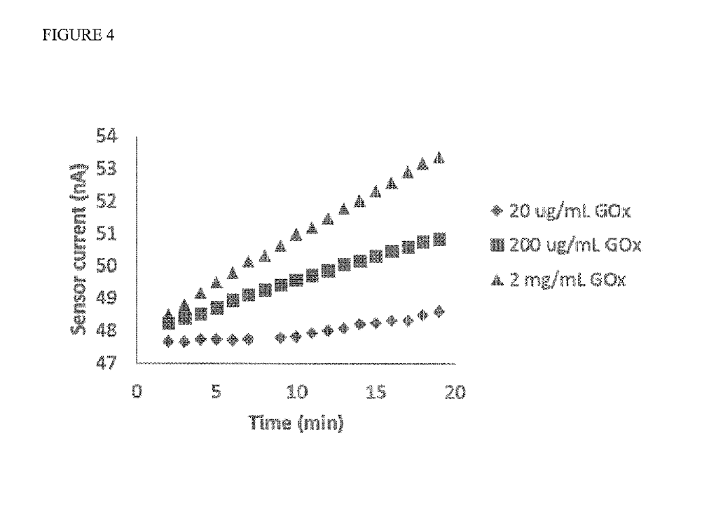

With reference to FIG. 4, both sensor current for a given time point and the rate of sensor current increase are proportional to concentration of the glucose oxidase enzyme.

Although the present invention has been described in terms of specific exemplary embodiments, it will be appreciated that various modifications, alterations and/or combinations of features disclosed herein will be apparent to those skilled in the art without departing from the scope of the invention as set forth in the following claims.

1. A method of testing a liquid sample for the presence of an analyte, the method comprising the steps of:
forming a mixture by contacting the sample with a composition comprising an oxidase for formation of hydrogen peroxide from the analyte, a fluorescent indicator precursor capable of forming a fluorescent indicator in the presence of an oxygen radical and an iron compound wherein the iron compound is dissolved in the mixture;
irradiating the mixture; and
measuring fluorescence from the fluorescent indicator.

2. A method according to claim 1 wherein hydrogen peroxide is formed from the analyte by an oxidase-catalysed reaction of the analyte.

3. A method according to claim 2 wherein the analyte is glucose and the oxidase is glucose oxidase.

4. A method according to claim 2 wherein the analyte is cholesterol and the oxidase is cholesterol oxidase.

5. A method according to claim 1 wherein the analyte undergoes one or more preliminary reactions to form a compound capable of oxidase-catalysed production of hydrogen peroxide.

6. A method according to claim 1 wherein the fluorescent indicator precursor is a fluorescein or a salt thereof.

7. A method according to claim 1 wherein the mixture does not comprise a quencher capable of quenching emission from the fluorescent indicator.

8. A method according to claim 1 wherein the sample is a biological liquid.

9. A method according to claim 1 wherein the liquid sample is brought into contact with the composition in a microfluidic device or lateral flow device.

10. A method according to claim 9 wherein the composition is provided in solid form in the microfluidic device or lateral flow device.

11. (canceled)

12. A method according to claim 1 wherein the sample is irradiated with visible light.

13. A method according to claim 1 wherein a concentration of the analyte is determined from the fluorescence measurement.

14. A method according to claim 1 wherein the mixture is formed by bringing the liquid sample into contact with the composition in solid form.

15. A method according to claim 1 wherein the liquid sample and composition are brought into contact in a sensor comprising a device for mixing the liquid sample and the composition; a light source for irradiation of the mixture; and a photodetector for detection of light emitted by the fluorescent indicator.

16. A method according to claim 15 wherein the device is a microfluidic device.

17. A method according to claim 1 wherein the iron compound is an iron (II) compound.

18. A composition comprising an oxidase for formation of hydrogen peroxide from an analyte; an iron compound; and a fluorescent indicator precursor capable of forming a fluorescent indicator in the presence of an oxygen radical, wherein the fluorescent indicator precursor is selected from the group consisting of: fluoresceins, rhodamines, coumarins, boron-dipyrromethenes, naphthalimides, perylenes, benzanthrones, benzoxanthrones; and benzothiooxanthrones.

19. (canceled)

20. A microfluidic device containing a composition according to claim 18 in solid form.

21. A lateral flow device comprising a composition according to claim 18 immobilised on a surface thereof.

22. A sensor comprising a device for mixing a liquid sample with a composition according to claim 18; a light source configured to irradiate the mixture; and a photodetector configured to detect light emitted by the fluorescent indicator.
